(19) United States
(12) Patent Application Publication
(10) Pub. No.: US 2009/0227528 A1
(43) Pub. Date: Sep. 10, 2009

(54) Pulmonary administration of an antithrombotic compound

(75) Inventor: Gerardus Martinus Theodorus Vogel, Oss (NL)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon, Oss (NL)

(21) Appl. No.: 11/664,803

(22) PCT Filed: Oct. 3, 2005
(86) PCT No.: PCT/EP05/54973

§ 371 Date: Apr. 5, 2007

(30) Foreign Application Priority Data
Oct. 6, 2004 (EP) 04104897.6

(51) Int. Cl. A61K 31/7064; A61K 31/702

(52) U.S. Cl. 514/32; 514/54

(57) ABSTRACT

The invention relates to the use of an oligosaccharide conjugated to a direct thrombin inhibitor for the manufacture of a medicament for the pulmonary delivery of said compound for the prevention or treatment of thrombosis or related disorders.

The invention relates to the use of oligosaccharides conjugated to a direct thrombin inhibitor for the manufacture of a medicament for the pulmonary delivery of said conjugates for the treatment or prevention of thrombosis or related disorders.

Further the invention relates to a pharmaceutical composition for said use.

Recently, the search for new compounds for the treatment and prevention of thrombosis and related disorders has resulted in the invention of new compounds which are oligosaccharides conjugated to a direct thrombin inhibitor. The new compounds are dual inhibitors having direct anti-thrombin (factor IIa) activity and anti-thrombin III (AT-III) mediated anti-Xa activity (WO99/65934; Bioorg. Med. Chem. Letters 1999; 9: 13-18).

Improvements of those compounds followed, the new dual inhibitors having a pharmacological profile with less side-effects and a half-life allowing once-a-day treatment (WO01/42262; J. Thromb. Haem. 2003; 1: 1945-1954).

Although the new antithrombotic compounds are clinically highly interesting compounds, they suffer from the drawback that—until now—the only effective route of administration is by injection (subcutaneous—s.c.-or intra venous—i.v.). As a result, the clinical use of these compounds is essentially limited to hospital settings or other situations where phycisians or other health care professionals are involved. Consequently, the compounds will find their use mostly in acute settings. In addition to the inconvenience of s.c. or i.v. injection, injection is further associated with side effects such as local irritation and ulceration. Chronic anticoagulant therapy is therefore practically not an option with these antitrombotic compounds.

Therefore, there is a need for a convenient noninvasive alternative route of administration for said antithrombotic compounds with a performance comparable to parenteral injection.

Heparin and low-molecular-weight heparins (LMWH) suffer from similar disadvantages with respect to the route of administration as described above for the new oligosaccharide conjugate compounds. Recently, it was reported that aerosol solid particle formation of heparins and LMWHs can result in the efficient pulmonary delivery of that type of molecules (WO 02/32406; PNAS Jun. 29, 2004; Vol. 101, No. 26, 9867-9872). It was discovered that pulmonary delivery depends on optimal formulation of dry aerosol particles. Absolute bioavailability of heparin, depending on formulation, was measured as 35-60% by the method described. A significant difference in the pulmonary delivery of heparin was observed between the inhalation of dry aerosol particles and the instillation of heparin in liquid form into the lungs. Instillation of liquid heparin into the lungs of rabbits resulted in low bioavailability and poor pharmacokinetics.

Surprisingly, in contrast to the finding that instillation of liquid heparin into the lungs is essentially ineffective, it has now been found that pharmaceutically acceptable solutions of the oligosaccharide compounds of WO99/65934 and WO 01/42262 may be used for the pulmonary delivery of said compounds. Unexpectedly high bioavailibility (>70%) was measured.

Therefore, the present invention relates to the use of a compound of the formula (I)

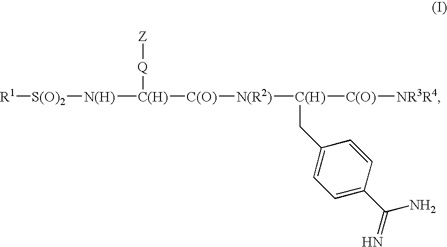

wherein R¹ is phenyl, naphthyl, 1,2,3,4-tetrahydronaphthyl, (iso)quinolinyl, tetrahydro(iso)quinolinyl, 3,4-dihydro-1H-isoquinolinyl, chromanyl or the camphor group, which groups may optionally be substituted with one or more substituents selected from (1-8C)allyl or (1-8C)alkoxy;
R² and R³ are independently H or (1-8C)alkyl;

### R⁴ is (1-8C)alkyl or (3-8C)cycloalkyl;

or R³ and R⁴ together with the nitrogen atom to which they are bonded are a nonaromatic (4-8)membered ring optionally containing another heteroatom, the ring optionally being substituted with (1-8C)alkyl or SO₂-(1-8C)alkyl;
Q is a spacer having a chain length of 10 to 70 atoms; and
Z is a negatively charged oligosaccharide residue comprising two to six monosaccharide units, the charge being compensated by positively charged counterions;
or a pharmaceutically acceptable salt thereof or a prodrug or solvate thereof,
for the manufacture of a medicament for the pulmonary delivery of said compound for the prevention or treatment of thrombosis or related disorders.

A preferred oligosaccharide conjugate for the use according to this invention is the compound of Formula (II)

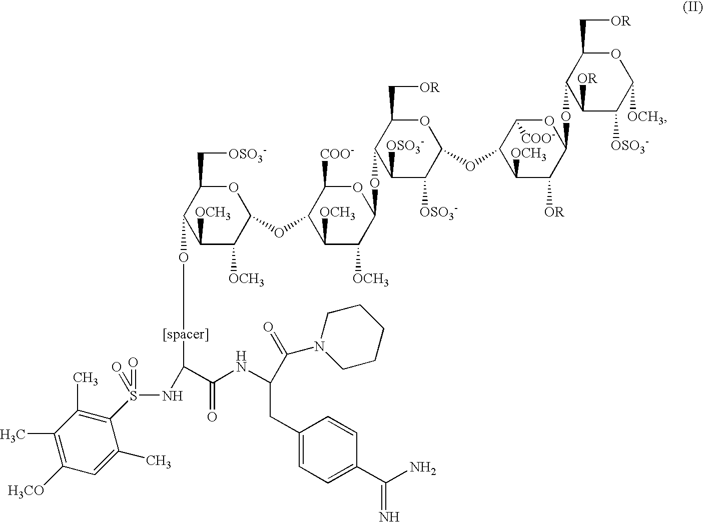

wherein R is independently SO₃⁻ or CH₃;
the spacer is a flexible spacer of a length of 13-25 atoms;
the charge of the pentasaccharide residue is compensated by positively charged counterions;
and the total number of sulfate groups in the pentasaccharide residue is 4, 5 or 6;
or a pharmaceutically acceptable salt, a prodrug or solvate thereof.

Particularly preferred is the compound of Formula (III)

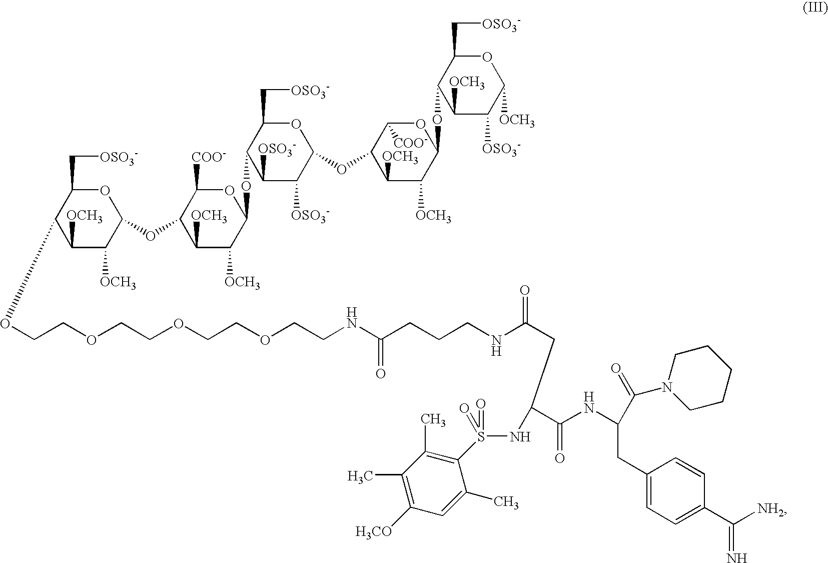

or a pharmaceutically acceptable salt, a prodrug or solvate thereof and specifically its octasodium salt, known by its code name Org 42675 (WO 01/42262; J. Thromb. Haem. 2003; 1: 1945-1954).

The use of the present invention comprises the use of the oligosaccharide conjugate either per se, without pharmaceutical auxiliaries, or a pharmaceutical composition thereof for pulmonary administration to a patient in need of treatment. The patient is a mammal and, usually, a human being.

The chemical structure of the spacer, as defined herein, is of minor or no importance for the anti-thrombotic activity of the compounds of the invention, it may however not be completely rigid. Highly flexible spacers are more preferred than others. Specific and highly suitable spacers are described in WO99/65934 and WO 01/42262 (incorporated by reference).

The term (1-8C)alkyl means a branched or unbranched alkyl group having 1-8 carbon atoms, for example methyl, ethyl, propyl, isopropyl, butyl, sec-butyl, tert-butyl, hexyl and octyl. Methyl and ethyl are preferred alkyl groups.

The term (1-6C)alkoxy means an alkoxy group having 1-6 carbon atoms, the alkyl moiety having the meaning as previously defined. Methoxy is a preferred alkoxy group.

The term (3-8C)cycloalkyl means a cycloalkyl group having 3-8 carbon atoms, being cyclopropyl, cyclobutyl, cyclopentyl, cyclohexyl, cycloheptyl or cyclo-octyl. Cyclopentyl and cyclohexyl are preferred cycloalkyl groups.

A positively charged counterion means H⁺, Na⁺, K⁺, Ca²⁺, and the like. Preferably the oligosaccharide conjugate compounds are in the form of their sodium salt.

The term prodrug means a compound of the invention in which the amino group of the amidino-moiety is protected, e.g. by hydroxy or a (1-6C)alkoxycarbonyl group.

Solvates according to the invention include hydrates.

The oligosaccharide conjugates of the invention may occur in the form of a free base, which may be isolated from the reaction mixture in the form of a pharmaceutically acceptable salt. The pharmaceutically acceptable salts may also be obtained by treating the free base of formula (I) with an organic or inorganic acid such as hydrogen chloride, hydrogen bromide, hydrogen iodide, sulfuric acid, phosphoric acid, acetic acid, propionic acid, glycolic acid, maleic acid, malonic acid, methanesulphonic acid, fumaric acid, succinic acid, tartaric acid, citric acid, benzoic acid, ascorbic acid and the like.

The oligosaccharide conjugate can be administered via the pulmonary route at several stages of the treatment according to the use of this invention. An embodiment of this invention is a method of preventing or treating thrombosis or related disorders in a mammal, comprising the administration to said mammal via the pulmonary route of a therapeutically effective amount of the oligosaccharide conjugate compound, or a pharmaceutically acceptable salt, a prodrug or solvate thereof. Preferably, the mammal is a human.

For use according to the invention, the oligosaccharide conjugate may be administered in a dosage of 0.001-1.0 mg per kg body weight per day. More preferably, the conjugate is administered at doses of between 0.2 mg and 5 mg per patient per day.

The oligosaccharide conjugate may be used alone or may be presented as a pharmaceutical composition. Thus, the present invention provides for a pharmaceutical composition for pulmonary administration comprising a therapeutically effective amount of said compound together with pharmaceutically acceptable auxiliaries and, optionally, other therapeutic agents. The term acceptable means being compatible with the other ingredients of the composition and not deleterious to the recipients thereof.

For administration according to the invention, the oligosaccharide conjugate may be presented alone, as dry particles, or as a pharmaceutical composition, either as dry particles further comprising pharmaceutically acceptable auxiliaries, or in a pharmaceutically acceptable liquid, optionally further comprising pharmaceutically acceptable auxiliaries. Preferably, the pharmaceutical composition is an isotonic solution of the oligosaccharide compound. The formulation is presented preferably in unit-dosages, in unit-dose or multi-dose containers, e.g. inhalation compositions for use in insufflators or inhalators in predetermined amounts.

Mixed with such pharmaceutically acceptable auxiliaries, e.g. as described in the standard reference, Gennaro et al., Remington's Pharmaceutical Sciences, (20th ed., Mack Publishing Company, see especially Part 5: Pharmaceutical Manufacturing, in particular also Chapter 50: Aerosols), and optionally by means of pharmaceutically acceptable liquids the oligosaccharide conjugate may be processed as an aerosol, or as a fluid composition, in the form of a solution, suspension, emulsion, or as a spray. Such composition may include a pulmonary delivery enhancer, e.g., a surfactant. Also aqueous suspensions, isotone saline solutions may be used, containing pharmaceutically acceptable dispersing agents and/or wetting agents, such as propylene glycol or butylene glycol.

A suitable aqueous formulation for use in this invention is a solution of the oligosaccharide conjugate in an isotonic phosphate-citrate buffer. Preferably, the pH is kept between 5 and 6. Alternative buffers may also be used with the same effect, such as—but not limited to—acetate and maleic acid, preferably in the pH range 5 to 6.

The pharmaceutical composition according to the invention may also be presented in the form of a veterinary composition, such compositions may be prepared by methods conventional in the art.

Also an embodiment of the present invention the use of the oligosaccharide conjugate according to the present invention, where a medicament comprising said compound is placed into a device which is suitable for administering pharmaceutical compositions via the pulmonary route. Particularly preferred is a device which is adapted for administering measured dosages.

Devices suitable for pulmonary delivery are well known in the art, e.g. inhalators, insufflators and ventilators for inhalation through the mouth or nasal passage, such as described in the standard reference, Gennaro et al., Remington's Pharmaceutical Sciences, (20th ed., Mack Publishing Company, see especially Part 5: Pharmaceutical Manufacturing, Chapter 50: Aerosols) or in the European Pharmacopeia 4.4 (04/2003:0671), J. Clin. Pharm. 2003, 56, 588-599; J. Clin. Pharm. 2003, 56, 600-612, etc. Also pressurized containers or dispensers may be considered, using a suitable propellant or nebulizer.

The invention further includes a pharmaceutical composition, as hereinbefore described, in combination with packaging material suitable for said composition, said packaging material including instructions for the use of the composition for the use as hereinbefore described.

The invention is further illustrated by the following examples. This should not be considered to be limiting in any way.

### EXAMPLE 1

### Pharmaceutical Formulation of Org 42675

A suitable aqueous pharmaceutical formulation for Org 42675 is a solution in an isotonic phosphate-citrate buffer comprising the following excipients:

Disodium hydrogen phosphate dihydrate: 10 mM
Citric acid monohydrate: 3 mM
pH=6.

### EXAMPLE 2

### Intratracheal/Intrapulmonary Administration of Org 42675

Comparative study between intravenous administration and intratracheal administration of Org 42765

The test was carried out in male Wistar rats of 300-400 gr. The rats were anaesthetised by inhalation of a mixture of O₂/N₂O/isoflurane, after which the right jugular vein was cannulated. Translumination of the neck enabled visibility of the vocal cords and an oral needle was inserted when the vocal cords were open after which 100 μl/300 gr body weight of a solution prepared from the pharmaceutical formulation of Example 1, diluted with an aqueous 0.9% sodium chloride solution, containing 300 nmol of Org 42675/ml was instilled at approximately 1 cm before the bifurcation of the trachea. The same solution and dose was also used to perform the i.v. study. After i.v. administration blood was sampled at 1, 6, 15, 30 and 60 minutes and after 2, 4, 7 and 24 hours. After intra-tracheal administration blood was sampled at 0.5-1-2-4-7 and 24 hours after administration. Blood was centrifuged and the plasma was siphoned off and stored at −20° C. until use. The concentration of Org 42675 was measured amidolytically with S2222 (Chromogenix, Chromogenics Ltd, Molndal, Sweden) by determination of the anti-Xa activity based on the method of Teien and Lie in the obtained plasma samples against a calibration curve which was made of the stock solution of 300 nmol/ml itself. (Teien A N, Lie M. Evaluation of an amidolytic heparin assay method increased sensitivity by adding purified antithrombin III. Thromb. Res. 1977, 10: 399-410). The concentration in the samples was expressed in nmol/ml and the kinetic parameters were calculated with the noncompartment model of WinNonlin (see Tables 1 and 2). After intratracheal administration the maximum concentration was already obtained at 0.5 hour after administration which was also the first time point of blood collection.

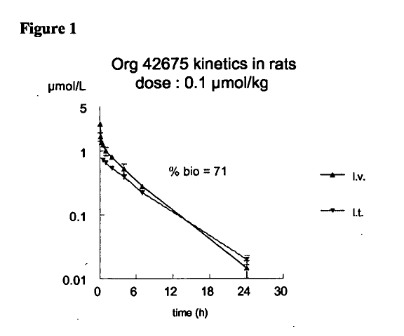

After i.v. administration an AUC_{inf} was obtained of 7.32±1.03 (mean±s.e.m. n=2) h.nmol/ml and after intratracheal administration the AUC_{inf} was 5.07±0.24 (mean±s.e.m. n=3) h.nmol/ml. This means that the bioavailability after intratracheal administration is 71±3% (see FIG. 1), where the bioavailability of i.v. administration is 100%.

### CONCLUSION

Org 42675 after intratracheal administration shows a pharmacokinetic performance which allows this route of administration to be used in home-medication and chronic anticoagulant therapy.

TABLE 1

| | |
|---|---|
| 100 nmol/kg | intravenous |

| Org 42675 | rat 1 | rat 2 | mean | s.e.m. |
|---|---|---|---|---|
| Cmax (nmol/ml) | 3.1815 | 2.6744 | 2.93 | |
| AUClast (h · nmol/ml) | 8.3527 | 6.2929 | 7.32 | 1.03 |
| AUCinf (h · nmol/ml) | 8.459 | 6.3462 | 7.40 | 1.06 |

TABLE 2
Org 42675 100 nmol/kg intratracheal

| | rat 1 | rat 2 | rat 3 | mean | ±s.e.m. |
|---|---|---|---|---|---|
| Tmax (h) | 0.5 | 0.5 | 0.5 | 0.5 | |
| Cmax (nmol/ml) | 0.64 | 0.72 | 0.81 | 0.77 | 0.05 |
| AUClast (h · nmol/ml) | 5.3968 | 5.205 | 4.6205 | 4.91 | 0.29 |
| AUCinf (h · nmol/ml) | 5.5283 | 5.3083 | 4.8327 | 5.07 | 0.24 |
| F (% bioavailabilty)* | 75.5 | 72.5 | 66.0 | 71.3 | 2.8 |

1-13. (canceled)

14. A method of preventing or treating thrombosis or related disorders in a mammal, comprising the administration to said mammal via the pulmonary route of a therapeutically effective amount of the compound of the formula (I) 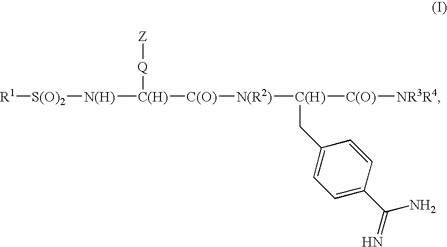
wherein R¹ is phenyl, naphthyl, 1,2,3,4-tetrahydronaphthyl, (iso)quinolinyl, tetrahydro(iso)quinolinyl, 3,4-dihydro-1H-isoquinolinyl, chromanyl or the camphor group, which groups may optionally be substituted with one or more substituents selected from (1-8C)alkyl or (1-8C)alkoxy;
R² and R³ are independently H or (1-8C)alkyl;
R⁴ is (1-8C)alkyl or (3-8C)cycloalkyl;
or R³ and R⁴ together with the nitrogen atom to which they are bonded are a nonaromatic (4-8)membered ring optionally containing another heteroatom, the ring optionally being substituted with (1-8C)alkyl or SO₂-(1-8C)alkyl;
Q is a spacer having a chain length of 10 to 70 atoms; and
Z is a negatively charged oligosaccharide residue comprising two to six monosaccharide units, the charge being compensated by positively charged counterions;
or a pharmaceutically acceptable salt thereof or a prodrug or solvate thereof.

15. The method according to claim 14, wherein the compound is the compound of Formula (II) 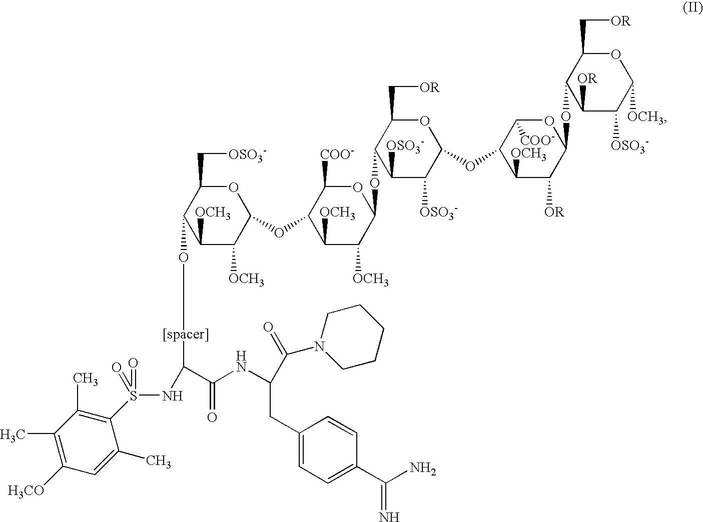
wherein R is independently SO₃⁻ or CH₃;
the spacer is a flexible spacer of a length of 13-25 atoms;
the charge of the pentasaccharide residue is compensated by positively charged counterions;
and the total number of sulfate groups in the pentasaccharide residue is 4, 5 or 6;
or a pharmaceutically acceptable salt, a prodrug or solvate thereof.

16. The method according to claim 15, wherein the compound is the compound of
Formula (III) 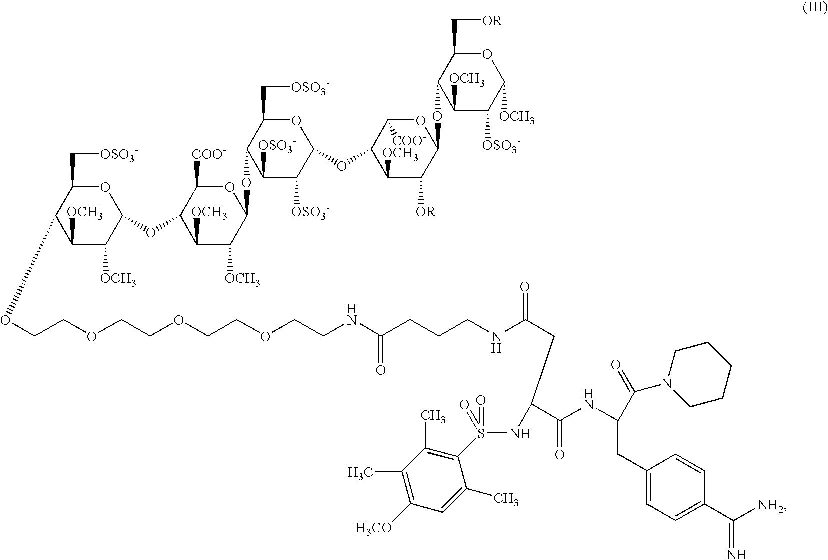
or a pharmaceutically acceptable salt, a prodrug or solvate thereof.

17. The method according to claim 16, wherein the compound of Formula (III) is in the form of its octasodium salt.

18. The method according to claim 14, wherein the compound is administered in the form of a pharmaceutical composition comprising a pharmaceutically acceptable auxiliary.

19. The method according to claim 18, wherein the pharmaceutically acceptable auxiliary is a pharmaceutically acceptable liquid.

20. The method according to claim 18, wherein the pharmaceutical composition is an isotonic solution of the compound.

21. The method according to claim 14, wherein the compound is in the form of a dry particle.

22. The method according to claim 18, wherein the pharmaceutical composition is provided in unit dosages.

23. The method according to claim 18, wherein the pharmaceutical composition is placed into a device which is suitable for administering a pharmaceutical composition via the pulmonary route.

24. The method according to claim 23, wherein the device is adapted for administering measured dosages.

25. A pharmaceutical composition for pulmonary delivery of the compound according to claim 14, or a pharmaceutically acceptable salt, a prodrug or solvate thereof, comprising a therapeutically effective amount of said compound together with a pharmaceutically acceptable auxiliary.

26. The pharmaceutical composition according to claim 25, wherein the pharmaceutically acceptable auxiliary is a pharmaceutically acceptable liquid.

27. The pharmaceutical composition according to claim 25, wherein the pharmaceutical composition is an isotonic solution of the compound.
